(19) United States
(12) Patent Application Publication
(10) Pub. No.: US 2007/0010481 A1
(43) Pub. Date: Jan. 11, 2007

(54) Solid State Irradiation of Hyaluronan-Based Solid Preparations, Its Derivatives And Mixtures In An Unsaturated Gaseous Atmosphere And Their Use

(75) Inventors: Endre A. Balazs, Fort Lee, NJ (US); Julie L. Heeter, East Stroudsburg, PA (US); Vadim Mitlitski, North Bergen, NJ (US); Ilana K. Mitlitski, North Bergen, NJ (US)

Correspondence Address: MATRIX BIOLOGY INSTITUTE, 725 RIVER ROAD, SUITE 205, EDGEWATER, NJ, 07020, US

(73) Assignee: MATRIX BIOLOGY INSTITUTE, Edgewater, NJ (US)

(21) Appl. No.: 11/160,754

(22) Filed: Jul. 7, 2005

(51) Int. Cl. A61K 31/728; C08B 37/00; C08G 63/91

(52) U.S. Cl. 514/54; 525/54.2; 536/53

(57) ABSTRACT

The present invention relates to a range of new products, production of biocompatible solid compositions based on hyaluronan and its derivatives using ionizing radiation in the solid state in the presence of an unsaturated gas under specified reaction conditions and the uses thereof.

### THE PRIOR ART

Hereafter, the term “hyaluronan” is used instead of the term “hyaluronic acid” as was suggested by E. A. Balazs et al. (E. A. Balazs, T. C. Laurent, R. W. Jeanloz, “Nomenclature Of Hyaluronic Acid”, Biomedical Journal, vol. 235(3), 1986, p.903) and the abbreviation HA for hyaluronan is used in the description of the invention.

Phillips (U.S. Pat. No. 6,610,810 and U.S. Pat. No. 6,841,644) discloses the processes for preparing a range of new products from dry biopolymers using ionizing radiation in the presence of an unsaturated gas.

### SUMMARY OF THE INVENTION

This invention provides a broad category of new biopolymers having dramatically improved properties in comparison with the starting material.

In carrying out the process of the invention for producing the new materials, it is preferable that hyaluronan is in dry, solid state, in an atmosphere of an unsaturated alkenic or alkynic gas, preferably acetylene. Before introducing the mediating gas to the reaction site, the site must be flushed to remove any traces of oxygen. The mediating gas is removed after the irradiation procedure and, therefore, the final product does not contain any of it.

After the system is flushed from oxygen and saturated with the mediating gas at atmospheric pressure, it is exposed to a source of ionizing irradiation which may be a either a radioactive isotope such as ⁶⁰Co(γ-rays) or radiation generated by a high energy (250 KeV to 10 MeV) electron accelerator or X-rays generated by the accelerator or any other suitable device.

The absorbed radiation dose may vary from 0.5 KGγ to 50 KGγ, depending on the structure of the polymer.

Following the irradiation, any residual gaseous mediating agent is removed from the system by aerating, and if necessary by application a vacuum process to the treated product. This will depend on the retention ability of the material for the gas which depends on the porosity of the solid system.

After irradiation of hyaluronan, two phases are usually formed—water or physiological salt solution soluble and water or physiological salt solution insoluble.

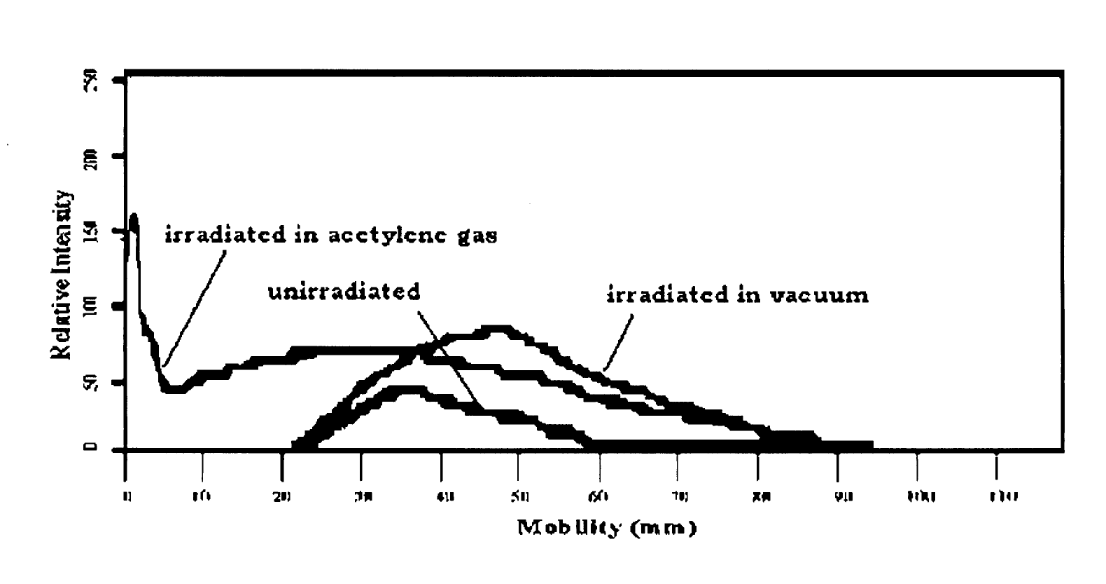

Molecular weight of soluble hyaluronan can be enhanced by irradiating the samples at a low dose such as 0.6 KGγ. FIG. 1 shows the densitometric scan of irradiated hyaluronan from agarose gel electrophoresis run compared to hyaluronan irradiated without acetylene and unirradiated control The distribution of the treated sample has both increases in the higher and lower MW portions of the curve compared to the control demonstrating the dual action of degradation as well as enhancement of the molecule.

As dose increases, density and degree of cross-linking of the insoluble portion increase as well. This is observed by the increase of HA concentration and amount of insoluble material created after irradiation.

The molecular packing of hyaluronan affects formation of the insoluble fraction. There is an increase in concentration and yield of insoluble portion created with treatment when the molecules of the starting material are packed closer.

When a mixture of different molecular weigh material is irradiated, it affects the stability of the final product. This mixture creates an insoluble fraction that degrades with time and its stability is dependent on temperature (4°, 25° or 37° C.). The rate of degradation depends on the amount and molecular weight of both portions in the mixture. Higher molecular weight portions degrade slower. Thus samples with a controlled dissolution rate can be created.

Using irradiation, hyaluronan can be cross-linked and copolymerized with other polysaccharides such as but not limited to cellulose and heparin and proteins such as but not limited to albumin and collagen.

The insoluble portion created during treatment is enhanced with multiple irradiations for most samples. After re-irradiation of various samples from previous different initial doses, enhancement of the solid portion occurs for most samples as demonstrated by an increase in insoluble material and insoluble material HA concentration. Samples that did not have any insoluble portion after the initial irradiation did produce an insoluble portion after re-treatment with high dose (≧13 kGy).

Annealing does not generally improve the resulting insoluble portion by way of increased HA concentration or increased amount of insoluble portion produced.

Irradiation with doses higher than 12 kGy has a benefit of producing sterile material.

The irradiated samples are biocompatible with various cell line growing in tissue cultures. Such irradiated material when introduced into the media and placed over cells growing on plastic or glass surfaces, did not cause any change in cells' growth or attachment.

The processes by which the hereinabove described products are obtained will now be discussed in detail in the following examples. These examples are given merely by way of illustration and are not intended to limit the invention as set forth in the claims.

Deflnition List 1

| Term | Definition |
|---|---|
| LMW HA | low average molecular weight HA |
| | preparations |
| MMW HA | medium average molecular weight HA |
| | preparations |
| Hylan A | water soluble material produced by |
| | treating hyaluronan-rich tissue sources |
| | with aldehydes prior to extraction. (Balazs |
| | and Leshchiner, 1989) |
| Hylan B | water insoluble material that is produced |
| | by treating hyaluronan or hylan A with |
| | divinyl sulphone under mild alkaline |
| | conditions. (Balazs and Leshchiner, 1989) |

### EXAMPLE 1

This example illustrates a typical procedure for the preparation and irradiation of a hyaluronan sample according to the present invention.

A dry sample of HA was placed in a small vial and capped with an open-top screw cap with a nylon filter membrane in place (10 μm pore size) between the cap and vial to allow an exchange of gases but prevent loss of sample. This vial was placed in an airtight bag sealed on three sides. The bag was evacuated of air and then pressurized with acetylene gas then heat sealed after a predetermined amount of exposure to the gas. The bagged and sealed sample was then exposed to 13-kGy dose of gamma radiation at room temperature. The bag was then opened under a fume hood to allow escape of the gas and then capped and stored at room temperature.

### EXAMPLE 2

This Example illustrates atypical procedure for preparation and irradiation of dry hyaluronan material where the dry sample is in the form of a string.

To prepare a string from HA, a concentrated solution of hyaluronan (2-3%) was extruded into isopropanol through 14-20 G cannula. The formed string-like precipitate was collected and air-dried at room temperature. The procedure described in Example 1 was repeated using the string as the dried material.

After irradiation and upon re-hydration, the insoluble fraction of the final material retained its shape of the string.

### EXAMPLE 3

This Example illustrates a typical procedure for preparation and irradiation of hyaluronan material where the dry sample is in the form of pellets.

To prepare pellets from HA, a concentrated solution of hyaluronan (2-10%) was precipitated as droplets with isopropanol without mixing. The formed precipitate was collected and air-dried at room temperature. The procedure described in Example 1 was repeated using pellets as a dried material.

After irradiation and upon re-hydration, the insoluble fraction of the final material retained the shape of pellets.

### EXAMPLE 3B

This Example illustrates a typical procedure for preparation and irradiation of hyaluronan material where the dry sample is in the form of small circular films.

To prepare discs from HA, a concentrated solution of hyaluronan (2-10% was added as droplets to a non-stick surface such as Teflon and the droplets were allowed to air dry into small, clear or cloudy round films. The procedure described in Example 1 was repeated using the film discs as a dried material.

After irradiation and upon re-hydration, the insoluble fraction of the final material retained its shape of the film which was transparent or opaque depending on the concentration of HA in the starting solution.

### EXAMPLE 4

This Example illustrates a typical procedure for preparation and irradiation of hyaluronan material where the sample is in the form of fibers.

To prepare fibers from HA, a diluted solution of hyaluronan (0.05-0.1%) was precipitated with isopropanol while mixing. The formed precipitate was collected as fibers and air-dried at room temperature. The procedure described in Example 1 was repeated using fibers as a dried material.

After irradiation and upon re-hydration, the insoluble fraction of the final material retained its shape of a fibrous mass.

### EXAMPLE 5

This Example illustrates a typical procedure for preparation and irradiation of hyaluronan material where the sample is freeze-dried.

To prepare freeze-dried material from HA, a concentrated solution of hyaluronan (2-3%) was freeze-dried using a lyophilizer. The procedure described in Example 1 was repeated using freeze-dried material.

After irradiation and upon re-hydration, the insoluble fraction of the final material retained its shape of the freeze dried mass.

### EXAMPLE 6

This Example illustrates a typical procedure for preparation and irradiation of hyaluronan material where the sample is in the form of particles.

To prepare particles from HA, a dilute solution of hyaluronan (0.05-0.1%) was precipitated with isopropanol. The formed precipitate was collected as particles and air-dried at room temperature. The procedure described in Example 1 was repeated using particles as a dried material.

After irradiation and upon re-hydration, the insoluble fraction of the final material retained its shape of the particles.

### EXAMPLE 7

This Example illustrates preparation of a clear film.

A previously irradiated material (at 1 kGy) was hydrated and the insoluble portion of the sample collected and homogenized then cast in a dish and air dried to create a clear, thin film. The procedure described in Example 1 was repeated using the dried film as the dried material. The resulting material was a water insoluble film that retained it's shape and had improved strength.

### EXAMPLE 7B

This Example illustrates preparation of a clear HA film.

A concentrated solution of hyaluronan (2-10%) was poured to a mold of a non-stick material such as Teflon. The fluid was allowed to slowly air dry to create a clear, thin film. The procedure described in Example 1 was repeated using the dried HA film as the dried material. The resulting material was a water insoluble film that retained it's shape.

### EXAMPLE 8

This Example illustrates preparation of a sample that after irradiation and rehydration degrades in solvents.

Two different molecular weight hyaluronans, one low MW and the other medium MW were co-precipitated as either pellets (example 3) or film discs (example 3b) with ratios of 1:4, 1:1, 2:1, 4:1, 8:1.

Subsequently, the resulting samples were irradiated according to the procedure described in Example 1. The resulting materials created an insoluble portion that degrades with time. Stability is dependent on temperature (4°, 25° or 37° C.). The rate of degradation depends on the amount and MW of both portions in the mixture. The higher the amount of medium MW or high MW portion, the slower the degradation. Results are summarized in the Tables 8a, 8b.

TABLE 8a
% sample as insoluble portion after 7 days at 37° C. in
excess saline. Sample = example 3B material with ratios
as follows: MMW = 1.8 M avg. MW LMW = 0.2 M avg. MW

| | % sample as insoluble portion. | | | |
|---|---|---|---|---|
| Ratio of MW in Sample | Day 0 | Day 1 | Day 2 | Day 7 |
| MMW/LMW 4:1 | 55 | 34 | 26 | 0 |
| MMW/LMW 1:1 | 35 | 28 | 0 | 0 |
| MMW/LMW 1:4 | 3 | 2 | 0 | 0 |

TABLE 8b
% sample as insoluble portion in samples tested
after 2× irradiation at 13 kGy

| Ratio of MW in Sample | Day 0 | Day 1 | Day 2 | Day 7 |
|---|---|---|---|---|
| MMW/LMW 4:1 | 48 | 29 | NA | 16 |
| MMW/LMW 1:1 | 39 | 25 | 23 | 14 |
| MMW/LMW 1:4 | 30 | 27 | 16 | 8 |

### EXAMPLE 9

This Example illustrates that irradiation can be carried out at different doses with the properties of final samples directly dependent on the dose.

The procedure described in Example 1 was repeated using various forms of dry HA. Irradiating doses were 0.6, 2, 4, 1 3, 28 and 35 kGy. Results are summarized in Table 9.

TABLE 9

| | | | |
|---|---|---|---|
| | | | HA concentration in |
| | | % Sample | insoluble material |
| Dose (kGy) | Form of Sample | as insoluble | (mg/ml) |

| | | | |
|---|---|---|---|
| 0.6 | pellets | 14 | NA |
| 2 | pellets | 42 | 7 |
| 4 | freeze dried | 29 | 23 |
| 13 | pellets | 41 | 14 |
| 28 | pellets | 48 | 29 |

### EXAMPLE 10

This Example illustrates preparation of copolymer samples of hyaluronan and other polysaccharides.

The procedure described in Example 1 was repeated where the dry sample was prepared by co-precipitating a mixture of HA and microcrystalline cellulose as fibers or HA and heparin as pellets. After microscopic investigation of the insoluble portion of the final samples, the cellulose sample appeared to have cellulose embedded in the HA cross-linked matrix while the heparin-HA copolymer appeared to be co-cross-linked uniformly.

### EXAMPLE 11

This Example illustrates preparation of copolymer samples such as hyaluronan and proteins.

The procedure described in Example 1 was repeated where a dry sample was prepared by co-precipitating a concentrated solution (2-3%) of HA and insoluble finely homogenized collagen as fibers, pellets or strings or HA and albumin as a mucin clot. After microscopic investigation of the insoluble portion of the final samples, cross-linking of hyaluronan with collagen was observed. The copolymerized sample appeared to contain networks of fibrous material different from a HA-only containingg sample. For the HA-albumin sample, the non-irradiated albumin clot dissolved in physiological saline in less than 24 hours. The irradiated mixture did not degrade for more than 14 days indicating a cross-link.

### EXAMPLE 12

This Example illustrates annealing after irradiation generally does not prevent degradation after irradiation. The effect varies with sample form and the average molecular weight of the hyaluronan dry material.

The procedure described in Example 1 was repeated where the dry sample is heated after irradiation for 1.5-2 hrs at 60° C. Samples were compared against identical samples without heating after irradiation for % sample as insoluble and HA content in the insoluble portion. Table 12 suggests that sample form plays a role in how heating will affect the irradiated sample.

TABLE 12
Annealing (heating) vs. no heat after irradiation, results
for various samples (all air dried)

| | | | | HA |
|---|---|---|---|---|
| | | | | content in |
| | | | | insoluble |
| | Starting material, | Form of | % sample as | portion |
| Sample ID | average MW | sample | insoluble | (mg/ml) |
| NH | hylan A, 5 M | fiber | 67 | 30 |
| | ppt'd in | | | |
| H | isopropanol | | 69 | 38 |
| NH | hylan A, 5 M | fiber | 72 | 29 |
| H | ppt'd in ethanol | | 59 | 18 |
| NH | hylan A, 5 M | freeze dried | 74 | 26 |
| H | freeze dried | | 73 | 21 |
| NH | MMW HA, 1.8 M | particles | 71 | 31 |
| | ppt'd in | | | |
| H | isopropanol | | 74 | 31 |

H = heated after irradiation at 65° C.,
NH = not heated after irradiation

### EXAMPLE 13

This Example illustrates a slightly different preparation of the dry pellet sample for irradiation. Instead of air-drying to remove excess isopropanol from the precipitation procedure, the sample was dried in the vacuum oven to completeness at 37-40° C. for 36-48 hrs to remove excess IPA as well as residual water. The procedure described in Example 1 was repeated and the insoluble portion of the sample was tested for % sample as insoluble and HA content in the insoluble portion. Table 13 shows the effect of sample dryness on the yield of the insoluble portion of the samples irradiated at 13 kGy.

TABLE 13

| | | Form | | calc. HA |
|---|---|---|---|---|
| | Starting material, | dry of | % sample as | content in |
| Sample ID | average MW | sample | insoluble | gel mg/ml |
| A | animal source HA, 1.2 M | pellet | 64 | 34 |
| O | | | 71 | 42 |
| A | hylan A, 5.6 M | pellet | 69 | 30 |
| O | | | 60 | 30 |
| A | hylan A, 1.3 M | fiber | 34 | 11 |
| O | | | 33 | NA |
| A | hylan A, 5.6 M | fiber | 61 | 8 |
| O | | | 60 | 11 |
| A | MMW HA, 1.2 M | fiber | 63 | 22 |
| O | | | 72 | 32 |
| A | hylan A, 5.6 M | string | 62 | 25 |
| O | | | 63 | 26 |
| A | MMW HA, 1.2 M | particles | 68 | 23 |
| O | | | 71 | 23 |

A = air dried,
O = oven dried at 37° C. under vacuum

### EXAMPLE 14

This Example illustrates use of various starting material for the sample preparation.

The procedure described in Example 1 was repeated where the dry sample was hylan HA of various MWs and different doses and sample forms. Results are summarized in the Table 14

TABLE 14

| | | | |
|---|---|---|---|
| | | insoluble HA | % sample as |
| | avg. MW | conc. | insoluble portion |
| dose (kGy) | of starting HA | (mg/ml) | (dry wt.) |

Pellets:

| | | | |
|---|---|---|---|
| 0.6 | 5 M | NA | 18 |
| 2 | 5 M | 8 | 44 |
| 13 | 5 M | 23 | 56 |
| 27 | 5 M | 29 | 48 |
| 29 | 0.3 M | 116 | 29 |

Fibers:

| | | | |
|---|---|---|---|
| 0.6 | 5 M | NA | 3 |
| 13 | 5 M | 8 | 61 |
| 13 | 1.3 M | 11 | 34 |
| 27 | 5 M | 5 | 34 |

Freeze Dried:

| | | | |
|---|---|---|---|
| 1 | 5 M | 16 | 27 |
| 4 | 5 M | 23 | 29 |
| 13 | 5 M | 21 | 74 |

String:

| | | | |
|---|---|---|---|
| 0.6 | 5 M | NA | 17 |
| 13 | 5 M | 21 | 57 |
| 29 | 5 M | 27 | 56 |

### EXAMPLE 15

This Example illustrates use of a different starting material for sample preparation.

The procedure described in Example 1 was repeated where the dry sample was bacterial HA of various sample forms and doses. Results are shown in Table 15.

TABLE 15

| | | insoluble HA | % sample as |
|---|---|---|---|
| | | concentration | insoluble portion |
| starting material | dose (kGy) | (mg/ml) | (dry weight) |
| Pellets | 0.6 | 0 | 0 |
| | 2 | 6 | 30 |
| | 27 | 41 | 61 |
| | 29 | 14 | 56 |
| Powder | 0.6 | 0 | 0 |
| | 29 | 16 | 77 |
| Freeze-dried | 1 | 0 | 0 |

### EXAMPLE 16

This Example illustrates use of a different starting material for sample preparation

The procedure described in Example 1 was repeated where the dry sample was HA of animal origin of various sample shapes and doses. The results are shown in the Table 16.

TABLE 16

| | | |
|---|---|---|
| | | % sample as |
| | insoluble HA | insoluble |
| | conc. | portion |
| dose (kGy) | (mg/ml) | (dry wt.) |

Pellets:

| | | |
|---|---|---|
| 0.6 | Not tested | 14 |
| 13 | 34 | 64 |
| 29 | 19 | 54 |

Granules:

| | | |
|---|---|---|
| 0.6 | Not tested | 8 |
| 13 | 27 | 70 |
| 27 | 28 | 66 |
| 29 | 14 | 76 |

String:

| | | |
|---|---|---|
| 0.6 | Not tested | 6 |

Fiber:

| | | |
|---|---|---|
| 13 | 22 | 54 |

### EXAMPLE 17

This example illustrates the effect of a secondary irradiation on dry samples.

After various samples were irradiated as per procedure described in the Example 1, they were subjected to γ-irradiation one more time at 13 kGy dose. Sample that could not produce insoluble portions after first irradiation, did produce insoluble portion after the secondary irradiation. Results are summarized in the Table 18.

### EXAMPLE 18

This Example illustrates the effect of a tertiary irradiation on dry samples.

For most, further increase in the yield of insoluble portion was observed after the samples were irradiated 3 times. Results are summarized in the Table 18.

TABLE 18

| | | | % Sample as Gel | |
|---|---|---|---|---|
| | | | (dry wt., mg | Gel conc. |
| Irradiation | dose, kGy | Sample type | gel/mg total) | mg/ml |
| 1st | 0.6 | hylan A fibers | 0 | 0 |
| 2nd | 13 | | 26 | 5 |
| 1st | 4 | | 0 | 0 |
| 2nd | 13 | hylan A fibers | 20 | 3 |
| 1st | 13 | | 17 | 3 |
| 2nd | 13 | hylan A fibers | 39 | 10 |
| 3rd | 13 | | 58 | 22 |
| 1st | 1 | | 0 | 0 |
| 2nd | 13 | FD* LMW HA | 42 | 13 |
| 3rd | 13 | | 49 | 21 |
| 1st | 4 | | 0 | 0 |
| 2nd | 13 | FD* bLMW HA | 39 | 12 |
| 3rd | 13 | | 58 | 23 |
| 1st | 13 | hylan A pellet | 39 | 12 |
| 2nd | 13 | | 58 | 16 |
| 1st | 30 | MMW HA pellet | 51 | 20 |
| 2nd | 13 | | 47 | 19 |
| 1st | 30 | LMW HA pellet | 46 | 21 |
| 2nd | 13 | | 46 | 20 |
| 1st | 13 | hylan A string | 16 | 4 |
| 2nd | 13 | | 33 | 9 |
| 1st | 13 | AD** hylan | 81 | 82 |
| | | B film | | |
| 2nd | 13 | | 85 | 125 |

FD*—freeze dried
AD**—air dried.

### EXAMPLE 19

This Example illustrates the biocompatibility of the soluble portion of irradiated materials using fibroblasts.

Samples were prepared as described in Example 3 using 2.5M average molecular weight HA and dose of 30 kGy. Concentration of the soluble portion after irradiation was 1% in media.

Adherent cell lines (fibroblasts L929, purchased from ATCC) were seeded in cell culture medium on plastic and allowed to attach. The media over the cells was removed and the soluble portion of the irradiated material was dispensed on top of the cells. The cells were incubated at 37° C., 5% CO₂ and observed on an inverted-phase microscope. The results showed that this material was not cytotoxic to the cells.

### EXAMPLE 20

This is another example that illustrates the biocompatibility of the soluble portion of irradiated materials using fibroblasts. An experiment was done as described in Example 19 except the concentration of the soluble portion was 0.1% in media.

The results showed that this material was not cytotoxic to the cells.

### EXAMPLE 21

This Example illustrates the biocompatibility of the soluble portion of irradiated materials using macrophages. The soluble portion of the irradiated material was prepared as described in Example 19.

The suspension cell line (macrophages TUR, purchased from ATCC) was suspended in the soluble portion of the irradiated material (1% concentration in media) and then dispensed into the plastic wells. The cells were incubated and observed as indicated in Example 19. The results showed that this material was not cytotoxic to the cells.

### EXAMPLE 22

This is another example that illustrates the biocompatibility of the soluble portion of irradiated materials using macrophages. An experiment was done as described in Example 21 except the concentration of soluble portion was 0.1% in media.

The results showed that this material was not cytotoxic to the cells.

### EXAMPLE 23

This Example illustrates the biocompatibility of the soluble portion of irradiated materials using stem cells.

Samples were prepared as described in Example 3 using 2.5M average molecular weight HA and dose of 30 kGy. Concentration of the soluble portion after irradiation was 1% in media.

Adherent cell lines (stem cells, NF-1, purchased from ATCC) were seeded in cell culture medium on plastic and allowed to attach. The media over the cells was removed and the soluble portion of the irradiated material was dispensed on top of the cells. The cells were incubated at 37° C., 5% CO₂ and observed on an inverted-phase microscope. The results showed that this material was not cytotoxic to the cells.

### EXAMPLE 24

This is another example that illustrates the biocompatibility of the soluble portion of irradiated materials using stem cells. An experiment was done as described in Example 19 except the concentration of soluble portion was 0.1% in media.

The results showed that this material was not cytotoxic to the cells.

### EXAMPLE 25

This Example illustrates the biocompatibility of both soluble and insoluble fractions of the irradiated materials using fibroblasts.

Samples were prepared as described in Example 3 with the exception of being precipitated directly in the wells of a 24-well plate (Becton Dickinson, cat. #353047) using high molecular weight hylan with starting concentrations of 1, 2 and 3%. After irradiation directly in the 24-well plate, samples were rehydrated either with media or a mix of media with 0.5% hylan solution.

Cells were seeded on the surface of the insoluble portions in a minimal amount of cell culture medium. The cells were incubated and observed as described in Example 19. The consistency of insoluble portions varied depending on the rehydration solution, however, none of the samples were cytotoxic to the cells.

### EXAMPLE 26

Procedure described in Example 25 was repeated using 2.5M average molecular weight hyaluronan as starting material.

The consistency of insoluble portions varied depending on the rehydration method, however, none of the samples were cytotoxic to the cells

Variations and modifications can, of course, be made without departing from the spirit and scope of this invention.

Having described out invention, what we desire to secure by Letters Patent and hereby claim is:

1. An improved process for modifying hyaluronan or its derivatives in solid or dry state by subjecting it to ionizing radiation in the presence of mediating gas and thereafter removing the mediating gas.

2. A process according to claim 1 where the modification to the treated hyaluronan include a) increasing the molecular weight to endow water binding ability, b) endowing the biopolymer to achieve desired viscosity or viscoelasticity, c) enabling biopolymer to be converted into insoluble gels of predetermined size and micromechanical properties.

3. A process according to claim 1 wherein the source of ionizing radiation is a γ-ray emitting radioactive isotope or X-rays or high energy ration generated by an electron accelerator.

4. A process according to claim 2 wherein the radioactive isotope is ⁶⁰Co.

5. A process according to claim 2 wherein hyaluronan or its derivatives are subjected to gamma radiation in the range of 0.6 to 50 kGy.

6. A process according to claim 1 wherein the unsubstituted alkynic gas is acetylene.

7. A process according to claim 1 wherein removal of any residual mediating gas is effected by aerating the system and optionally, additionally applying vacuum.

8. A process according to claim 1 wherein the modified biopolymer retains the same degree of biocompatibility as the starting material.

9. A process according to claim 1 wherein the process is conducted at atmospheric pressure.

10. A process according to claim 1 where the biopolymer is hyaluronan of animal origin.

11. A process according to claim 1 where the biopolymer is hyaluronan of bacterial origin.

12. A process according to claim 1 where the biopolymer is hylan.

13. A process according to claim 1 where staring material is a pellet.

14. A process according to claim 1 where staring material is a string.

15. A process according to claim 1 where staring material is a film.

16. A process according to claim 1 where staring material is air-dried.

17. A process according to claim 1 where the biopolymer is a mix of hyaluronans of different molecular weights.

18. A process according to claim 1 where the biopolymer is a mix consisting of hyaluronan and a protein(s).

19. A process according to claim 1 where the biopolymer is mix consisting of hyaluronan and another polysaccharide(s).

20. A process according to claim 1 wherein the process described is repeated twice or more times.

21. A process according to claim 1 where the final product is sterile.

22. A modified naturally occurring biocompatible biopolymer, which is hyaluronan or its derivatives that are produced by a process comprising subjecting, said polymer in the solid or dry state to an ionizing radiation in the presence of mediating gas and thereafter removing the mediating gas.

23. A modified biopolymer according to claim 22 wherein the radioactive isotope is ⁶⁰Co.

24. A modified biopolymer according to claim 22 wherein hyaluronan or its derivatives are subjected to gamma radiation in the range of 0.6 to 50 kGy.

25. A modified biopolymer according to claim 22 wherein the unsubstituted alkynic gas is acetylene.

26. A modified biopolymer according to claim 22 wherein removal of any residual mediating gas is effected by aerating the system and optionally, additionally applying vacuum.

27. A modified biopolymer according to claim 22 wherein the modified biopolymer retains the same degree of biocompatibility as the starting material.

28. A modified biopolymer according to claim 22 wherein the process is conducted at atmospheric pressure.

29. A modified biopolymer according to claim 22 where the biopolymer is hyaluronan of animal origin.

30. A modified biopolymer according to claim 22 where the biopolymer is hyaluronan of bacterial origin.

31. A modified biopolymer according to claim 22 where the biopolymer is hylan.

32. A modified biopolymer according to claim 22 where staring material is a pellet.

33. A modified biopolymer according to claim 22 where staring material is a string.

34. A modified biopolymer according to claim 22 where staring material is a film.

35. A modified biopolymer according to claim 22 where staring material is air-dried.

36. A modified biopolymer according to claim 22 where the biopolymer is a mix of hyaluronans of different molecular weights.

37. A modified biopolymer according to claim 22 where the biopolymer is a mix consisting of hyaluronan and a protein(s).

38. A modified biopolymer according to claim 22 where the biopolymer is mix consisting of hyaluronan and another polysaccharide(s).

39. A modified biopolymer according to claim 22 wherein the process described is repeated twice or more times.

40. A modified biopolymer according to claim 22 where the final product is sterile.

41. A modified biopolymer that has various forms.

42. A modified biopolymer that has different levels of hydration.

43. A modified biopolymer that has improved physical characteristics.

44. A modified biopolymer where its properties are affected by water content and average molecular weight and polydispersity of the starting material and/or dose of irradiation.

45. A modified biopolymer, which is biocompatible and non-toxic.

46. A modified biopolymer that can be used to grow cells on the surface or within the insoluble portion.

47. A modified biopolymer to be used for implantation of cells to grow tissues in vitro.

48. A modified biopolymer that can be used for anti-adhesion.

49. A modified biopolymer that can be used for wound dressing.

50. A modified biopolymer that can be used as hemostatic agent.

51. A modified biopolymer that can be used for controlled release drug delivery systems.
